(19) United States
(12) Patent Application Publication
(10) Pub. No.: US 2023/0223104 A1
(43) Pub. Date: Jul. 13, 2023

(54) GENOME RECONSTRUCTION METHOD USING WHOLE GENOME DATA

(71) Applicant: GIST (Gwangju Institute of Science and Technology), Gwangju (KR)

(72) Inventors: Hyunju LEE, Gwangju (KR); Yeonghun LEE, Gwangju (KR)

(73) Assignee: GIST (Gwangju Institute of Science and Technology), Gwangju (KR)

(21) Appl. No.: 18/152,037

(22) Filed: Jan. 9, 2023

(30) Foreign Application Priority Data
Jan. 10, 2022 (KR) 10-2022-0003453

(51) Int. Cl. G16B 20/00; G16B 45/00

(52) U.S. Cl. CPC G16B 20/00; G16B 45/00

(57) ABSTRACT

Disclosed is a genome reconstruction method using whole genome data. According to the present invention, the genome reconstruction method reduces detection errors by converting a nucleotide sequence having a structural variation into a graph form, and then reconstructing the graph so that the structural variation and the copy number variation have consistent values. Thereafter, the genome arrangement form was restored by constructing a haplotype graph using heterozygous single nucleotide polymorphism information and then finding an Eulerian path with a minimum entropy value.

### CROSS-REFERENCE TO RELATED APPLICATIONS

This application claims the priority of Korean Patent Application No. 10-2022-0003453 filed on Jan. 10, 2022, in the Korean Intellectual Property Office, the disclosure of which is incorporated herein by reference.

### BACKGROUND OF THE INVENTION

### Field of the Invention

The present disclosure relates to a genome reconstruction method using whole genome data.

### Description of the Related Art

Whole genome data refers to data that provides nucleotide sequences of the entire DNA of an individual subject. The human genome consists of 3 billion nucleotide sequences, and in the case of cancer cells, there are genetic variations different from those of normal cells. It is important for personalized treatment to accurately identify different genetic variations for each individual cancer.

However, it is a very difficult task to accurately identify the variations in cancer cells by analyzing 3 billion nucleotide sequences, and particularly, a rearranged chromosome structure which has been observed in cancer cells under a microscope in the past has not yet been identified at the level of a single nucleotide sequence. Therefore, there is a need for an algorithm capable of analyzing and identifying the whole genome.

Cancer cells undergo numerous changes in their DNA, from point mutation to DNA rearrangement to ultimately generate a complex cancer-associated genome. Iterative chromosome structural variations (SVs), such as translocation, fold-back inversion, chromothripsis, homogeneously staining regions (HSR; represents iterative gene amplification), and double minute (DM; extrachromosomal DNA) as well as simple SVs, such as tandem duplication, deletion, inversion and insertion that have been extensively studied, are associated with oncogenesis.

Traditional karyotyping techniques, such as G-banding and fluorescent in situ hybridisation (FISH), may confirm existence of complex SVs from either a derivative chromosome (a byproduct of recombination of a multiple chromosome with an intact centrosome) or a marker chromosome (an abnormal chromosome of which the genome has not been identified). However, due to limited resolution (to 5 Mb), it is not possible to accurately identify complex SVs on derivative or marker chromosomes using a standard karyotyping technique.

High-throughput sequencing has improved the understanding of SV by resolving genomic changes at a single nucleotide level. An early-stage method has been developed to detect SV using discordant and split reads in sequencing data. However, these methods have limited detection capability for SV to identify an SV breakpoint in a local genomic region, and do not interpret SV in the entire genomic region. Recently, several methods have been developed to integrate genomic information such as cancer purity and ploidy, total copy number alteration (CNA), allele-specific CNA and haplotype information in order to identify SVs. These methods use graph-based representation of the rearranged cancer genome, but do not analyze actual karyotypes of linear and/or circular chromosomes and thus do not generate karyotypic topologies such as HSR, DM, or chromothripsis. The global reconstruction of the genomic karyotypes in cancer may find out the basis of cancer development and evolution.

The above-described technical configuration is the background art for helping in the understanding of the present invention, and does not mean a conventional technology widely known in the art to which the present invention pertains.

### SUMMARY OF THE INVENTION

An object of the present disclosure is to provide a method for reconstructing cancer genome karyotypes based on complex topological analysis while providing a haplotype graph-based representation.

A graph-based framework of the present disclosure uses input SV call, unmapped read, read-depth information and single nucleotide polymorphism (SNP) to configure a breakpoint graph in order to model linkage between genomic segments at a genome-wide scale.

The present disclosure classifies a rearrangement topology and derives cancer genome karyotypes from a haplotype graphical output (FIG. 1).

In addition, the analytical potential of the method of the present disclosure is shown by comparing existing tools using simulation data and cancer cell line data. In addition, using WGS data from The Cancer Genome Atlas (TCGA) and European Genome-phenome Archive (EGA), the present disclosure reconstructs the karyotypes of cancer cells and differentiates private and shared SVs from primary and metastatic cancer cells to evolve tumors.

According to an aspect of the present disclosure, there is provided a genome reconstruction method using whole genome data, and the genome reconstruction method may include steps of 1) detecting an initial structural variation of a genomic segment of a whole genome sequence; 2) constructing a breakpoint graph from the genomic segment and the structural variation; 3) constructing an allele-specific breakpoint graph; 4) constructing a haplotype breakpoint graph; and 5) enumerating Eulerian paths by pairing edges of the breakpoint graph.

In the 1) detecting of the initial structural variation of the genomic segment of the whole genome sequence, the structural variation may be indicated as head-to-head (HH), head-to-tail (HT), tail-to-head (TH) or tail-to-tail (TT) according to a direction of breakpoint adjacencies.

In step 2), graph nodes may include a head node (Sb) and a tail node (Sₜ), and graph edges may include a segment edge (Eₛ), a reference edge (Eᵣ), and an SV edge (Eᵥ).

The segment edge may link a head node and a tail node of an nth genomic segment, and the multiplicity of the segment edge may indicate the copy number (CN) of the genomic segment.

The reference edge may link an nth tail node and an n+1th head node between the nth and n+1th genomic segments, and represent adjacency between adjacent genomic segments present in the reference genome.

The SV edge may represent adjacency between genomic segments which are not present in the reference genome.

The 2) constructing of the breakpoint graph from the genomic segment and the structural variation may be performed by a) performing local copy number segmentation; b) predicting an integer copy number (CN) by integer programming; and b) determining edge multiplicity by the integer programming.

The a) performing of the local copy number segmentation may include determining a breakpoint consisting of the following two terms:

a likelihood term describing how well a model with breakpoints fits read-depth data; and

a parameter or penalty term of controlling the number of breakpoints and preventing over-segmentation.

The b) predicting of the integer copy number may include sequentially substituting the integer copy number according to a high probability in an integer measurement model from the read-depth of the genomic segment.

The edge multiplicity may be indicated by the multiplicities of a segment edge, a structural variation edge, and a reference edge, and the 2) constructing of the breakpoint graph from the genomic segment and the structural variation may further include d) removing a structural variation with edge multiplicity of 0 after the c) determining of the edge multiplicity by the integer programming.

The 2) constructing of the breakpoint graph from the genomic segment and the structural variation may further include iteratively performing steps a) to d) until the structural variation with the edge multiplicity of 0 is not detected.

The 3) constructing of the allele-specific breakpoint graph may further include dividing an integer CN into an allele-specific copy number (ASCN) by integer programming.

The dividing of the integer CN into the allele-specific copy number (ASCN) by the integer programming may be performed using a negative binomial model for different depths of the SNP.

The allele-specific breakpoint graph may be constructed based on the allele-specific copy number (ASCN).

The allele-specific breakpoint graph may consist of a balanced node and an imbalanced node.

The 4) constructing of the haplotype breakpoint graph may include defining a haplotype segment from the allele-specific breakpoint graph of step 3); phasing balanced heterozygous SNP and imbalanced heterozygous SNP; and constructing a haplotype breakpoint graph by integer programming.

The enumerating of the Eulerian paths may include pairing breakpoint graph edges using a multiway tree structure.

The enumerating of the Eulerian paths may include prioritizing an Eulerian path with minimum entropy.

According to another aspect of the present disclosure, there is provided a recording medium recording a program executable by a computer to perform the genome reconstruction method using the whole genome data.

The recording medium may be a CD-ROM, a DVD-ROM, a portable storage device, a ROM, a RAM, or forms of transmission via Internet.

Information recorded on the recording medium may be expressed in the form of a compiled binary file, a text file, or a shell script.

According to the genome reconstruction method of the present disclosure, detection errors were reduced by converting a nucleotide sequence having a structural variation into a graph form, and then reconstructing the graph so that the structural variation and the copy number variation have consistent values. Thereafter, the genomic arrangement form was restored by constructing a haplotype graph using heterozygous single nucleotide polymorphism information and then finding an Eulerian path with a minimum entropy value. According to the genome reconstruction method of the present disclosure, the genetic variation detection error was greatly reduced, the genomic arrangement form of a cancer cell line was restored to a single nucleotide sequence level, and the genetic variation detection accuracy of the present disclosure was significantly improved compared to Illumina Algorithm Manta, an international genomic sequencing specialized company.

In addition to the above-described effects, specific effects of the present disclosure will be described together with explanation of specific matters for carrying out the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other aspects, features and other advantages of the present invention will be more clearly understood from the following detailed description taken in conjunction with the accompanying drawings, in which:

FIG. 1: a) Head-to-tail (HT), tail-to-head (TH), tail-to-tail (TT) and head-to-head (HH) SVs for two chromosomes (chrA and chrB) and two haplotypes (hap1 and hap2) are expressed; b) Construction of breakpoint graph—a breakpoint graph consisting of nodes and segment edges (blue and green boxes), reference edges (black lines) and SV edges (colored lines) is constructed after reads of whole genome sequencing data (WGS) are aligned. The iteration consisting of three steps is displayed in the first box. log2ratio represents a normalized copy number (CN) and the number below a node is an integer CN (low-confident CNs are displayed in gray). False-positive edges are changed to gray edges after integer programming. When the graph converges (first iteration), reads (green) supporting deletion are remapped and a split parameter λ is updated before the second iteration; c) Construction of allele-specific graph—ASCN is measured for each segment using a negative binomial model and imbalanced segments are divided into allele segments (light blue and blue in the case of chrA, and light green and blue in the case of chrB). Balanced segments (grey) are maintained in the same state in the breakpoint graph; d) Construction of haplotype graph—allelic segments are haplotypes that constructs the haplotype graph using HMMs (blue and green arrows) to be phased; e) The problem of finding an Eulerian path of the haplotype graph is to finally reconstruct a cancer genome from alignment data. Here, the example has a unique path.

FIG. 2: F-measures for five variant calling categories (SVs, SVCNs, CNA breakpoints, integer CNs, and ASCNs) and switch error rates for a haplotype were compared with those of other variant calling tools. Comparison was made in a condition with a control (somatic variant) and a condition without a control (total variations including gametic and somatic variations). An x-axis represents haplotype coverage (3× to 20×) and represents the average number of reads aligned to nucleotides of the haplotype.

FIG. 3: A haplotype graph consists of nodes and segment edges and reference edges and SV edges (black and colored lines, respectively) of two haplotypes (green and blue boxes).

The copy number of allele segments is indicated by the color intensity (1 to 5 copies). SVs included in the karyotyping are indicated as D (deletion), TD (tandem duplication), T (translocation), FB (foldback inversion) and C (complex SV). a to e: Interchromosomal SV sets across karyotypes and chromosomes of cell lines H292(a), A549(b), H226(c) and HeLa (d, e) are shown.

FIG. 4: Karyotype possibility of BRCA and GBM from The Cancer Genome Atlas (TCGA). Here, SV clusters in the haplotype graph are indicated by square brackets. The SV clusters are denoted as HSR, HSR/DM, DM and CT with cancer-associated genes (red texts) amplified simultaneously. A patient identifier of the TCGA is indicated at the top of each karyotype. Iterative cycles suggestive of DM formation are indicated by circles; a) commonly rearranged chromosome 17 with interchromosomal SV clusters in BRCA. The SV cluster of BRCA is linked to a chromosomal arm or telomere terminus to form a derivative chromosome with HSR, and CT is usually accompanied therein. Each chromosome terminus with the SV cluster is connected by a gray line. b) DM formation of generally rearranged chromosomes 4, 7, 12 and GBM. DM below the chromosomal karyotypes is indicated with cancer-associated genes amplified in the SV cluster.

FIG. 5: a) Bar plot of SVs and SV clusters found in metastatic and recurrent breast cancers from the European Genome-phenome Archive (EGA) data set. Four cancer types studied included primary tumour, local relapse, sync. axillary LN metastasis and distant metastasis. Cancer types were classified in the listed order and patients were classified as having primary or metastatic evolution according to accumulative pattern of SVs. b) Patient PD4252 showed karyotypic evolution of chromosomes 1 and 9. c) Patient PD4820 showed karyotypic evolution of chromosome 6. d) Patient PD11460 showed karyotypic evolution of chromosomes 8 and 11. e) Patient PD9193 showed karyotypic evolution of chromosomes 11 and 14. In b to e, EGA patient identifiers are indicated at the top of each karyotype. Cancer-associated genes are indicated in red texts.

### DETAILED DESCRIPTION OF THE PREFERRED EMBODIMENT

Hereinafter, the present disclosure will be described in more detail through Examples and Experimental Examples.

However, these Examples are just to help in the understanding of the present disclosure and the scope of the present disclosure is not limited to these Examples in any meaning.

In this specification, a case where a part “comprises” an element will be understood to imply the inclusion of stated elements but not the exclusion of any other elements unless explicitly described to the contrary.

Meanwhile, the operation of the present invention to be described below may be performed by a processor such as a central processing unit (CPU) or a graphics processing unit (GPU), and the processor may include at least one physical element comprising application specific integrated circuits (ASICs), digital signal processors (DSPs), digital signal processing devices (DSPDs), programmable logic devices (PLDs), field programmable gate arrays (FPGAs), controllers, and micro-controllers.

1. InfoGenomeR reconstructs candidate genomic karyotypes.

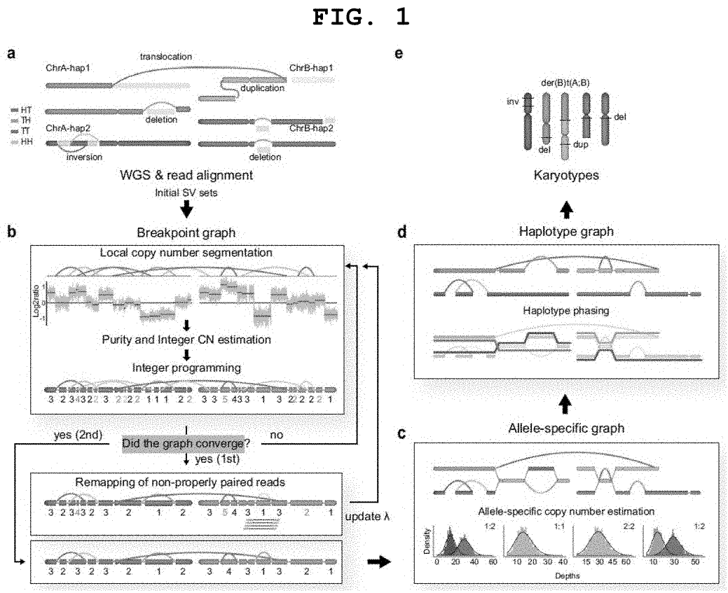

First, InfoGenomeR evaluates all reads in a WGS data set, generates initial structural variation (SV) calls using the discordant and split reads analysis tool (FIG. 1A), and perform initial copy number (CN) segmentation using a read-depth analysis tool. Thereafter, the initial SV and the CN breakpoints are used to construct an initial breakpoint graph of a local genomic segment. The breakpoint graph consists of nodes, segment edges, reference edges, and SV edges. The next three-step iteration updates the initial breakpoint graph.

In each iteration, (i) the local genomic segment is refined, (ii) the integer CN of the genomic segment is estimated using purity and ploidy, and (iii) integer programming of the CN balance condition determines the edge multiplicities of the breakpoint graph and removes SVs with multiplicity of 0.

Each iteration restarts with a SV set without containing SVs with multiplicity of 0, the CN segmentation is performed without a previous false-positive SV breakpoint, and the integer CN of the segment is recalculated. The iteration is performed until the graph converges (stopped when no SV with multiplicity of 0 is observed). The iteration consists of primary and secondary iterations according to segmentation parameters, and the CN segment is more generally merged with a neighbor CN segment in the secondary iteration than in the primary iteration. In an intermediate step between the primary and secondary iterations, non-properly paired discordant or unmapped reads are remapped to a candidate neighbor sequence of an imbalanced node (b of FIG. 1). Thereafter, candidate adjacencies supported by the reads are generated and the secondary iteration completes the breakpoint graph.

Next, the integer CN is divided into an allele-specific copy number (ASCN) using a negative binomial model for different read depths of heterozygous SNP, and an expectation-maximisation (EM) algorithm is used to estimate a parameter.

In a CN balanced condition including the ASCN, the integer programming constructs an allele-specific breakpoint graph and then adjusts the phase of an imbalanced heterozygous SNP sequence (c of FIG. 1).

A genomic segment containing balanced heterozygous SNP phase-adjusted using a hidden Markov model, and a final haplotype breakpoint graph is constructed (d of FIG. 1).

The Eulerian path may be enumerated to obtain a candidate genome by pairing breakpoint graph edges using a multiway tree structure with a minimum-entropy search.

Eventually, InfoGenomeR generates candidate karyotypes of cancer cells at the haplotype level (e of FIG. 1).

2. InfoGenomeR has performance superior to other variation calling methods.

Based on the simulated data set, the performance of InfoGenomeR was evaluated on eight different tools in six limited variant call categories, SV, SV copy number (SVCN), CNA breakpoints, integer CN, ASCN and haplotype. These six categories were evaluated for whole and somatic modes. Different methods were compared to detect variation in each category. The performance of each method was evaluated before integration into InfoGenomeR.

The present inventors performed a quadruple cross-validation for each haplotype range, where the selected parameter or threshold was determined by enumerating the defined value range. To compare InfoGenomeR with JaBbA, a recent graph SV caller, we ran JaBbA was executed using the same SV common set inputs (DELLY2, Manta, and NovoBreak) as used for InfoGenomeR. Since JaBbA is sensitive to input purity and ploidy hyperparameters, the purity and ploidy estimation of InfoGenomeR for JaBbA input was used. Various hyperparameter settings for JaBbA together with the JaBbA recommendations were tested and the most suitable setting for SV detection was selected. Performance metrics for precision and recall for each variant calling category were determined.

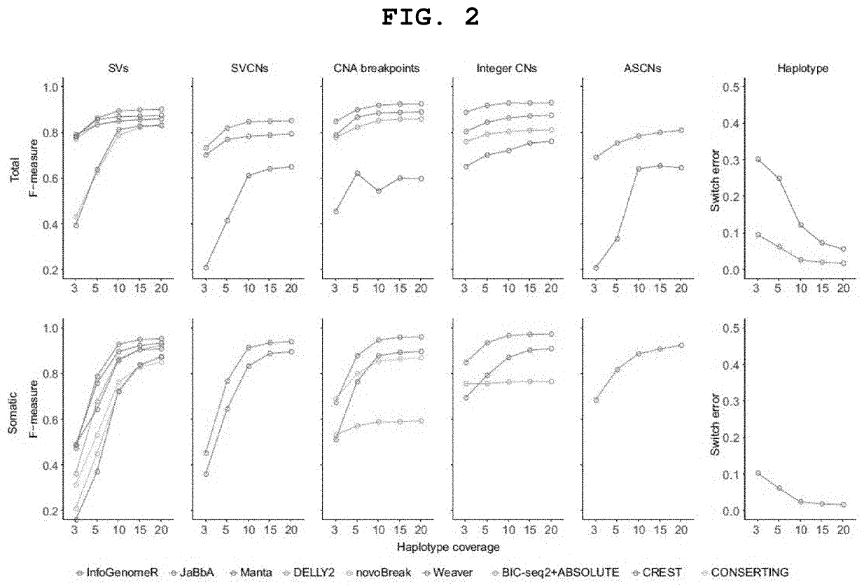

When comparing three methods (DELLY2, Manta, and NovoBreak) using discordant and split reads with three methods (CONSERTING, Weaver, and JaBbA) using both discordant/split read and read depth, InfoGenomeR achieved the highest whole (precision, 0.987, recall, 0.825) and somatic (precision, 0.981, recall, 0.919) SV calling performance in a 15× haplotype range (FIG. 2). Also, JaBbA produced the second best result for SV calling. As the result of the present inventors, it was confirmed that the integration strategy of InfoGenomeR gave improved performance compared to private SV tools (DELLY2, Manta and NovoBreak) and that InfoGenomeR is superior to another graph SV caller JaBbA.

CONSERTING and Weaver used both the discordant/split reads and the read depth, but are inferior to DELLY2 and Manta. On the other hand, InfoGenomeR showed higher precision while maintaining the recovery rate in the initial SV call. In addition, InfoGenomeR remapped non-properly paired reads to imbalanced nodes to detect SVs at an intermediate stage, resulting in improved 2.8% in recovery rate for somatic SVs. Since the read depth integration with SV may be sensitive to the variant size, the performance was compared based on variant size. In other words, InfoGenomeR remained robust regardless of the variant size and showed the highest precision and recall compared to all other test methods. Finally, when InfoGenomeR, Weaver and JaBbA were compared in terms of SVCN detection, InfoGenomeR showed better performance (FIG. 2).

In the case of a CNA breakpoint call, InfoGenomeR showed more improved performance than BIC-seq2 due to a local segmentation strategy (FIG. 2). In particular, InfoGenomeR predetermines CNA breakpoints using the initial SVs (first iteration), discovers CNA breakpoints where candidate SVs exist (intermediate stage), and reduces the wrong breakpoints in the split area by increasing split parameters (second iteration). Local segmentation solves the balance between noise filtering and actual variation calling to provide the highest precision and recall. The CONSERTING uses a local segmentation approach similar to InfoGenomeR, but has lower performance. On the other hand, the Weaver showed the lowest performance and was sensitive to variation size and purity. JaBbA showed second best performance at the CNA breakpoint. Next, the performance of detecting integer CN and ASCN of the segment region was compared based on positive proof (defined as >90% of the segment region with the same status, including the actual number of copies). In the case of the integer CN, Weaver may detect whole integer CNs, but may not detect somatic integer CNs using a germline coverage control. In addition, the capacities to detect total and somatic integer CNs were compared by combining BIC-seq2 with ABSOLUTE. JaBbA was compared with InfoGenomeR for both total and somatic integer CNs. InfoGenomeR showed improved performance compared to the combination of BIC-seq2 and ABSOLUTE, and surpassed JaBbA to achieve the best performance in the integer CN detection. In the case of ASCN, InfoGenomeR showed an F-measure of 0.799 (15×), which is 14% higher than an F-measure of Weaver. In the case of somatic ASCN, InfoGenomeR showed an F-measure value of 0.907 (15×). Since ASCN is dependent on the number of SNPs, small gamete variants (<10 kb) were observed to cause a bottleneck. InfoGenomeR showed F-measures of 0.940 (total variants) and 0.925 (somatic variants) for large ASCNs (>100 kb).

For haplotype estimation, a switch error rate between the actual haplotype and the inferred haplotype was measured based on the whole or somatic breakpoint graph. InfoGenomeR showed error rates of 1.98% and 1.87% for the whole mode and the somatic mode, respectively (15×) (FIG. 2), and a small decrease in the error rate for the somatic mode was caused with the higher accuracy of somatic ASCN estimation.

InfoGenomeR showed better performance for haplotype estimation than Weaver by obtaining an advantage of better ASCN estimation than Weaver.

InfoGenomeR was evaluated on five different tools using a GRCh38-based simulation data set to compare the performance according to human reference genome versions. Compared to the GRCh37-based simulation data set, the performance difference between SV callers was reduced. The reduction in performance difference may be due to improved mappability of GRCh38. InfoGenomeR and JaBbA for whole SVs and InfoGenomeR and Manta for somatic SVs showed the best performance in sequence, respectively. InfoGenomeR and JaBbA showed similar performance for whole CNA breakpoint calls. The performance difference between variation calling methods may be reduced by improving the mappability of the GRCh38 reference, but the high performance of InfoGenomeR was still valid for GRCh38 reference. Considering these results, InfoGenomeR showed better performance than other variation calling methods in all limited variation call categories for both GRCh37 and GRCh38 references.

3. Validation using Cancer Cell Lines

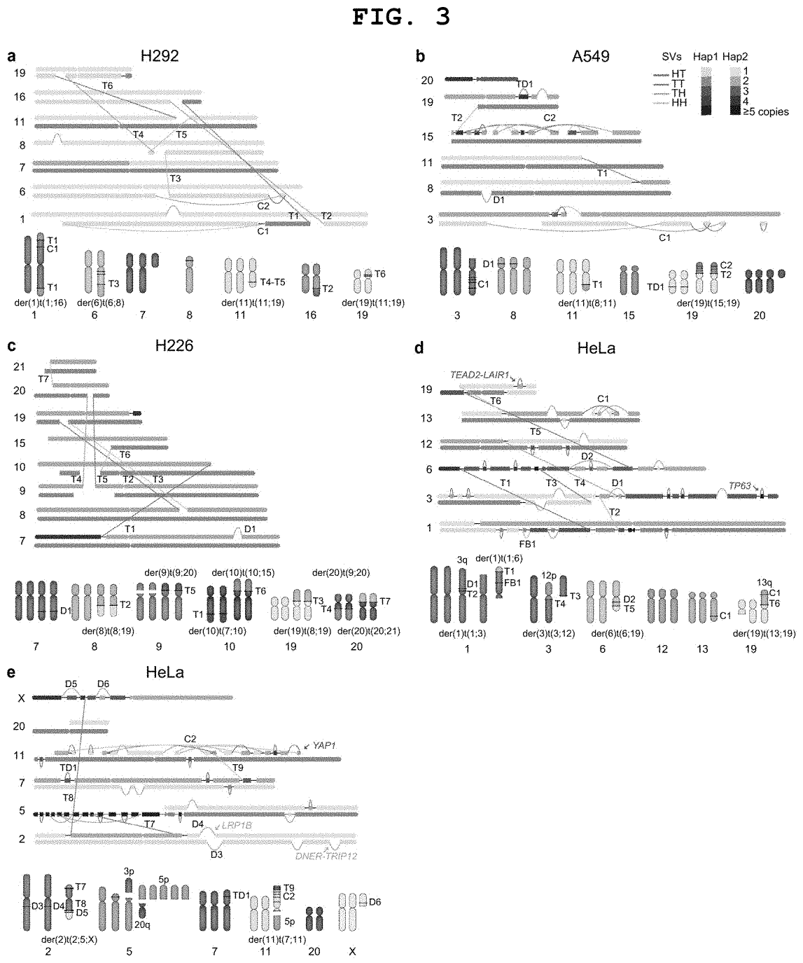

To evaluate the performance of InfoGenomeR, WGS data of three lung cancer cell lines H292, A549 and H226 and a HeLa cell lines with well-known karyotypes were analyzed. The present inventors constructed a haplotype graph for each cell line (FIG. 3). Since the graph includes several karyotype possibilities according to the alternative Eulerian path, a karyotype with minimum entropy was selected from candidate karyotypes for verification of the karyotype. The karyotypes and chromosomal terminuses reconstructed by InfoGenomeR were compared with karyotypes and chromosomal terminuses found in m-FISH. InfoGenomeR identified 62.5%, 50.0%, 53.3% and 40% of interchromosomal translocations in m-FISH (Table 1). Most of the unidentified translocations were found in centromeric or telomeric regions.

TABLE 1
Performance of karyotype reconstruction for cancer cell lines

| Cell | | Translocation | | Karyotype | |
|---|---|---|---|---|---|
| line | Ploidy | Precision | Recall | Precision | Recall |
| H292 | diploidy | 1.000 | 0.625 | 0.800 | 0.870 |
| | | (5/5) | (5/8) | (40/50) | (40/46) |
| A549 | triploidy | 1.000 | 0.500 | 0.924 | 0.968 |
| | | (2/2) | (2/4) | (61/66) | (61/63) |
| H226 | tetraploidy | 0.875 | 0.583 | 0.775 | 0.863 |
| | | (7/8) | (7/12) | (69/69) | (69/80) |
| HeLa | triploidy | 0.890 | 0.400 | 0.680 | 0.828 |
| | | (8/9) | (8/20) | (53/78) | (53/64) |

With respect to correctly identified interchromosomal translocations, InfoGenomeR may detect breakpoints and complex SV types such as chromothripsis that cannot be marked with m-FISH at base-pair resolution of haplotypes. The H292 cell line showed chromoplexes (rearrangement chains) between chromosomes 6, 8, 11, and 19 (T3-T6 and C2 in a of FIG. 3). As the result, there are der(6)t(6;8), der(11)t(11;19) and der(19)t(11;19). The A549 cell line was triploidy and showed chromothripsis on chromosomes 3 and 15 (C1 and C2 in b of FIG. 3, respectively). der(19)t(15;19)x2 generated from the chromothripsis of chromosome 15 was reconstructed. In addition, the present inventors reconstructed the karyotype of the tetraploid H226 cell line with balanced translocations t(8;19) and t(9;20) (T2-3 and T4-5 in c of FIG. 3, respectively) and imbalanced translocations t(7;10), t(10;15) and t (20;21) (T1, T6 and T7 in c of FIG. 3, respectively). A derivative chromosome was duplicated, which suggested that translocation was followed by whole genome duplication (WGD).

For the HeLa cell line, 9 translocations were identified, of which 8 translocations were accordant with translocations identified by m-FISH. The discordant translocation was present between 3p and the centromeric region of the chromosome and represents the centromeric noise (T3 of d of FIG. 3). In particular, the present inventors reconstructed representative HeLa-derived chromosomes [der(1)t(1;3), der(12)t(3;12) and der(19)t(13;19)] using used InfoGenomeR at the base-pair resolution (d of FIG. 3). The result showed that chromosome 11 had excessive SV with loss of heterozygosity (LOH), which suggested the derivative chromosome of der(11)t(7,11) (e of FIG. 3).

Furthermore, the present inventors found TP63 and MYC tandem duplications with cancer-level amplification and local YAP1 amplification in der(11)t(7;11) of the HeLa cell line; this amplification recurs in cervical cancer. In addition, the present inventors analyzed changes in expression levels according to SV using accordant RNA-seq data (methods). The present inventors detected homozygous exonic deletions in LRP1B tumor suppressor gene and four head-to-tail or tail-to-tail gene fusions (DNER-TRIP12, SLC12A3-NLRC5, KLHDC4-SLC7A5 and TEAD2-LAIR1). This data was validated using discordant reads of accordant RNA-seq data. As confirmed by the reconstructed karyotypes, gene expression in the derivative chromosome was upregulated in proportion to the increased copy number. In summary, the reconstructed genome was supported by previously published reports in cervical cancer and RNA expression data.

4. InfoGenomeR May Specialize Complex SVs and Karyotypes of Cancer.

It was shown that InfoGenomeR may construct the karyotypes of cancer cells, and the present inventors applied InfoGenomeR to various data sets of breast invasive carcinoma (BRCA, n=90), glioblastoma multiforme (GBM, n=37) and variant serous cystadenoma (OV, n=47) derived from TCGA. InfoGenomeR identified average 223, 124 and 275 somatic SVs in the BRCA, GBM and OV data sets, respectively, of which >20% were complex SVs. The present inventors performed clustering analysis of these complex SVs in the haplotype graph to define SV clusters as a set of closely rearranged local segments. The present inventors further classified SV clusters of BRCA, GBM and OV into three amplification types: (1) HSR (SV cluster with high amplification (>10 copies) linked to chromosomal arm); (2) HSR/DM (SV cluster having cycles with high amplification and 5 or more multiplicities linked to chromosomal arm); and DM (SV cluster having cycles with at least 5 multiplicities without linkage to chromosomal arm). The HSR/DM amplification type indicates an SV cluster with unclear distinction between HSR and DM or simultaneous presence of HSR and DM. The present inventors also classified deletion chromothripsis, an SV cluster interspersed with LOH.

Next, the present inventors examined data for each cancer type individually.

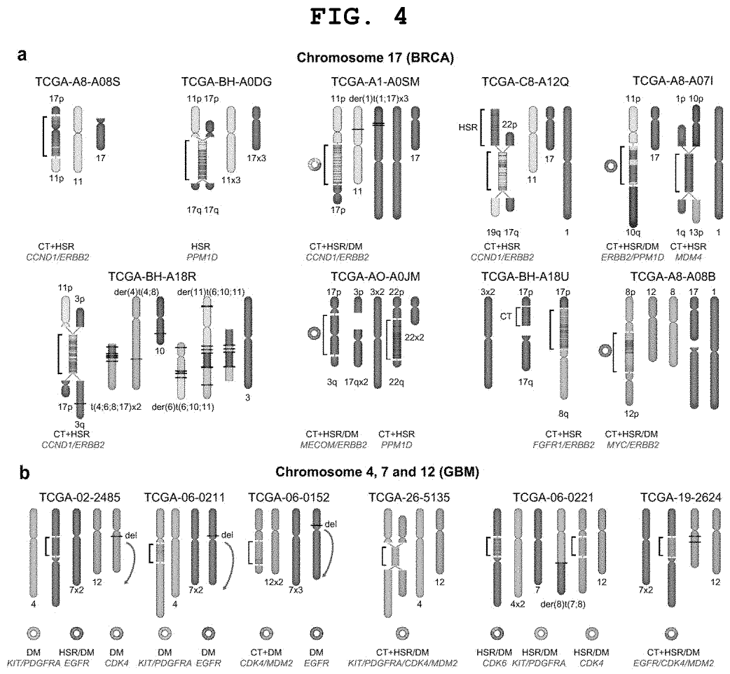

In the BRCA data set, the present inventors derived a karyotype structure of 9 patients rearranged on chromosome 17 (a of FIG. 4). In the result of the present inventors, chromosomes 11 and 17 were the most commonly rearranged chromosomes and exhibited HSR or HSR/DM type SV clusters.

In addition, HSR and HSR/DM are accompanied by CT to create a derivative chromosome.

The result of the present inventors showed that interchromosomal SVs cause frequent clustering of ERBB2 on chromosome 17 together with other amplified oncogenes in HSR and HSR/DM (a of FIG. 4). In addition, CCND1 on chromosome 11 was most commonly clustered with ERRB2, followed by MECOM, FGFR1 and MYC. In summary, these findings provide a karyotype evidence for colocalization of oncogenes and suggest that CT is associated with HSR and HSR/DM, which are frequently observed in BRCA.

The main feature of oncogene amplification in DM, the GBM data set, was observed in 16.2% (6/37) of the samples (b of FIG. 4). DM was not present on a chromosome with excision or CT, and the remaining segments produce LOH and were linked to each other. The present inventors observed HSR/DM in 59.4% (22/37) of samples. Most of the SV clusters were classified into HSR/DM because DM required stringent conditions not linked to the chromosomal arm. Major GBM oncogenes, that is, CDK, MDM2, KIT, PDGFRA and EGFR, were amplified in HSR/DM and DM. In addition, CDK4 and MDM2 were the most frequently clustered partners on chromosome 12, whereas KIT, PDGFRA and EGFR showed interchromosomal clustering with CDK4 and MDM2. In particular, the amplification is high and local, suggesting the possibility of DM shown to be developed through a mechanism different from the HSR observed in BRCA.

OV is characterized by a cluster of arm-level CNAs and fold-back inversions suggesting a common breakage-fusion-bridge (BFB) cycle on chromosome 19 (n=7, 14.9% of OVs). The fold-back inversions induce inverted repeats that produce HSR and is strongly associated with poor prognosis in OV. The present inventors observed HSR with a BFB cycle (fold-back inversions ≥5) in a derivative chromosome with interchromosomal SVs in which BRD4 and CCNE1 are frequently amplified on chromosome 19. HSR with BRCA-like CT together with the BFB cycle was also observed on the derivative chromosome with BRD4 and CCNE1 amplification, which suggests that other mechanisms may be involved in the amplification.

5. Application of Multi-Sample WGS Data to Elucidate Tumor Evolution

Tumor evolution was examined in the context of single nucleotide variants (SNVs) and CNAs. However, differentiation between individual and shared SVs in primary or metastatic cells has been less thoroughly examined due to a discordant SV calling rate between false-positive SVs and multiple samples. InfoGenomeR was applied to multi-sample WGS data of locally recurrent or metastatic breast cancer samples (methods) downloaded from EGA (EGAD00001002696). The present inventors analyzed 34 tumor samples from 15 patients with primary and/or metastatic lesions.

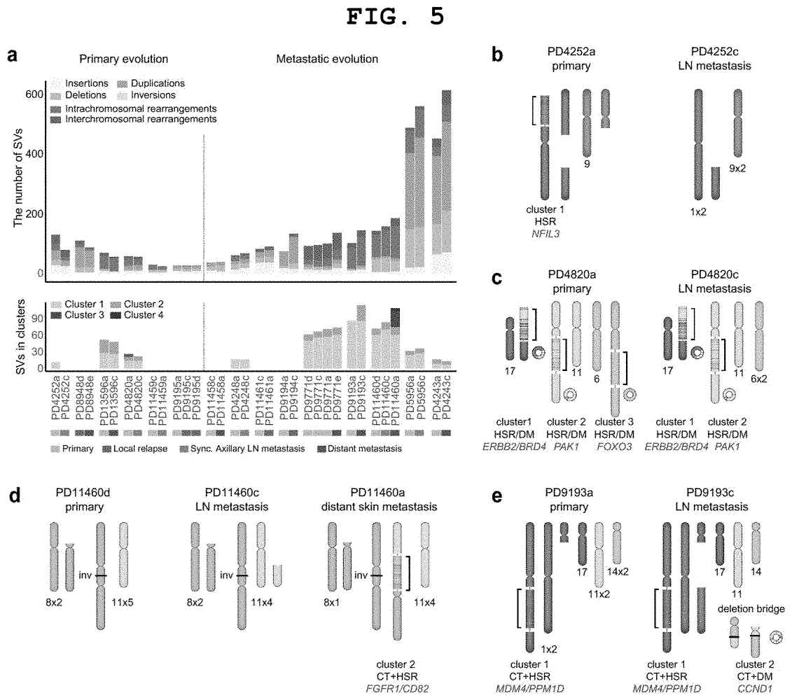

6 patients showed higher private SV accumulation in the primary tumor than in the metastatic tumor (a of FIG. 5). Two (PD4252 and PD4820) of these patients showed new SV clusters in the primary tumor (FIGS. 5B and 5C). The patient PD4252 had a LOH deletion of 9q in the primary tumor, and the remaining segments were integrated into chromosome 1 with a LOH of 1p to form an HSR with NFIL3 amplification (PD4252a). The patient PD4820 had HSR/DM amplified with ERBB2 and BRD4 and HSR amplified with PAK1, which passed during lymph node (LN) metastasis (PD4820c). In the primary tumor, an inverted repeat indicating FOXO3 amplification was generated together with a new SV cluster (Cluster 3). These results indicate the acquisition of SV clusters in the primary tumor after LN metastasis. Minor subclones without SV clusters may be metastasized to LN, but no subclone CNAs were observed in the primary tumor, and thus this idea was ignored.

Other 9 patients showed either newly generated SV clusters or developed SV clusters compared to the primary tumor in metastatic tumors, which indicates metastatic evolution (a of FIG. 5). Two (PD11460 and PD9193) of these patients showed new SV clusters in the metastatic tumors. The present inventors found divergence evolution between metastatic lesions and primary tumors in the patient PD11460. In addition, the loss of 11p evolved only in metastatic LN tumor (PD11460c), whereas a new cluster (PD11460 cluster 2) between chromosomes 8 and 11 was generated in metastatic skin tumor. This new cluster caused HSR on the derivative chromosome with local amplification of FGFR1 and CD82 (>10 copies) (d of FIG. 5). In the primary tumor (PD9193a) in the patient PD9193, there was an SV cluster (PD9193 cluster 1) inherited by a metastatic LN tumor (PD9193c) (e of FIG. 5). A new DM (cluster 2) was generated to be isolated from chromosome 11 with CT, which had a locally amplified CCND1 (>30 copies). The remaining segments of chromosome 11 were translocated to chromosome 14 by a small deletion bridge. These results show the evolution process of generation of HSR and DM accompanying CT.

<Example>

1. Detection of Initial Structural Variations

Variant callers DELLY2, Manta and novoBreak used in Example were used with basic parameters to detect (whole or somatic) initial SVs with or without controls. Low-quality SVs defined as <3 variantions or mapping quality <20 supporting reads were filtered. Breakpoints in SVs were aligned according to the chromosome and coordinate order of a reference sequence, and SVs were annotated as head-to-head (HH), head-to-tail (HT), tail-to-head (TH), or tail-to-head depending on a direction of breakpoint adjacencies to genomic segments. -Annotated as tail-to-tail. The head and the tail were 5′ and 3′ coordinates of a reference genome, respectively. The SV sets of an private SV caller were integrated into the input of InfoGenomeR. The breakpoints predicted by the SV caller may be different for the same SV (if breakpoints in the SVs overlap by less than 100 bp, the SVs were considered the same SV), and when the SV sets are integrated, one of the corresponding breakpoints was selected empirically.

2. Construction of Breakpoint Graph

InfoGenomeR constructs a breakpoint multi-graph G (S, E) from genomic segments and SVs. In a node set S had two types, a head node Sₕ and a tail node Sₜ, and each representing the head and tail sides of the genomic segment. In a breakpoint graph, a genomic segment i^{th} was represented by a pair of head and tail nodes Sⁱₕ and Sⁱₜ. An edge set E had three types of a segment edge Eₛ, a reference edge Eᵣ, and an SV edge Eᵥ. The segment edge linked the head node sⁱₕ and the tail node sⁱₜ of the genomic segment i^{th}, and the multiplicity of the segment edge indicated the CN of the genomic segment. The reference edge linked a tail node sⁱₜ and a head node sⁱ⁺¹ₕ between i^{th} and a segment i+1th to indicate adjacency between adjacent genomic segments present in the reference genome. On the contrary, the SV edge represented new adjacencies between genomic segments that did not exist in the reference genome. The following iteration procedure was used to construct the breakpoint graph:

Iteration Step 1—Local CN Segmentation

InfoGenomeR segmented a genomic region using current SV breakpoints.

Then, local CN segmentation was performed using a major penalty parameter λ and BIC-seq2 in the pre-segmented region and a copy ratio between the observed and expected number of reads (compared to a control, if available) in the genomic region was measured.

BIC-seq2 used in Example was a read-depth analysis tool that determined breakpoints using Bayesian information criteria consisting of the following two terms; a likelihood term describing how well a model with breakpoints fitted the read-depth data, and a parameter or penalty term of controlling the number of breakpoints and preventing over-segmentation.

The parameter λ adjusted the penalty term and prevented excessive breakpoints as λ was higher.

InfoGenomeR used different λ in the iteration of rounds 1 and 2, and the iteration of round 2 used a higher penalty to merge wrongly segmented regions without SV evidence. In the current analysis, parameter values of bin size=100, initial λ=1 and final λ=16 were used for simulation data. Cancer cell line data showed a higher noise level than the simulation data, and parameter values of bin size =100, initial λ=1, and final λ=2000 were used for the cancer cell line data, wherein the reconstructed karyotype was accordant well with an m-FISH karyotype. The same parameters used for the cancer cell line data were used for TCGA and EGA data.

Iteration Step 2—Prediction of Purity and Integer CN

A duplication rate of the genomic segment was determined by local CN segmentation, and cancer purity (p) and ploidy (τ) were estimated using ABSOLUTE. The end sides of the genomic segment were denoted as head and tail nodes, and a copy ratio and an integer CN of the genomic segment at a node s (head or tail) were denoted as copyratio(s) and CN(s), respectively. The copyratio(s) was fitted to a Gaussian mixture model, and each component was a Gaussian distribution representing an integer CN state (q) with an average copy ratio of m_{q}={qp+2(1−p)}/D. Here, q was an integer CN (0, 1, 2, . . . ) in the cancer genome and D=pτ+2(1−p) was an average ploidy of cancer and normal cells.

ABSOLUTE used in the Example estimated the cancer purity and ploidy from the copy ratio, and the integer copy number CN(s) was allocated according to the highest posterior probability of the integer copy number in the Gaussian Mixture Model. Since ABSOLUTE allocated a predefined maximum integer CN when copyratio(s) was greater than an estimation limit, in this case, the present inventors calculated non-integer CN'(s) satisfying a copy ratio equation, copy ratio(s)={CN'(s)p+2(1−p)}/D. Then, CN(s) was allocated to [CN'(s)] by rounding off the non-integer CN'(s).

Perturbation was performed on a low-confident segment and the integer CN was determined in the next integer programming step. The segment of the node s was defined with low-confidence as follows.

(1) posterior probability of integer CN, p(CN(s)) <0.95, (2) segment size (s) <50 bp or no depth information in s (unmappable region) or (3) high CN|CN(s)−CN'(s)|>0.35.

The purity estimation was iterated during the

InfoGenomeR iteration step, and the final purity was determined in the last iteration.

Iteration Step 3—Integer Programming for Finding Edge Multiplicities

An optimal breakpoint graph representing a cancer genome was reconstructed, wherein the edge multiplicitues met a CN balance condition (Equation 1).

The CN balance condition ensured an Eulerian path from one telomere to the other telemere for each linear chromosome.

The multiplicity of the edge e was represented by μ(e) and the segment edge, the SV edge (there may be multiple SVs) and the reference edge adjacent to the node s were presented by eₛ(s), Eᵥ(S), and eᵣ(s), respectively.

The multiplicity μ(eₛ(s)) of the segment edge was a sum of the multiplicity μ(eᵣ(s)) of the reference edge and the multiplicity μ(eᵥ(s)) of the SV edge for the node s.

$$μ ( {e}_{s} ( s ) ) = μ ( {e}_{r} ( s ) ) + {\sum }_{v ∈ {E}_{v} ( s )} μ ( v ) , ∀ s ∈ S \backslash telomeric ends \qquad [ Equation 1 ]$$

The multiplicities of edges satisfying the CN balance condition were determined by integer programming. In the case of a confident segment edge, the multiplicity was given by the integer CN of the segment, which was an estimate value of the previous copy (constant if the segment of the node was confident).

To determine the multiplicities of variable edges (reference and SV edges, and low-confident segment edges), first, a interrelated node subset S_{related} was found, and then an integer programming problem (Equation 2) was solved to find the multiplicity of edges adjacent to the interrelated node but independent of other subsets. The interrelated subset was defined inductively to include an adjacent node Adj (s) in a start node sₛₜₐᵣₜ, and may be found by a Broadth-First Search (BFS) method.

When the BFS encountered a confident node, propagation in a segment edge direction was stopped (an adjacent node Adjs(s) by the segment edge was not included, whereas another adjacent node Adj_{r,v}(s) by the reference and SV edges was included).

After S_{related}={Sₛₜₐᵣₜ} was constructed for any sₛₜₐᵣₜ, S_{related} was extended as follows:
Adj_{r,v,s}(s) ⊂ S_{related} if CN(s) is low-confident, ∀s ∈ S_{related} Adj_{r,v}(s) ⊂ S_{related} if CN(s) is confident, ∀s ∈ S_{related}

When a constant integer CN state of the segment was given in each interrelated subset, the multiplicities of the reference edge and the SV edge were determined with small perturbation of the integer CN of the low-confident segment. An optimization problem was defined to satisfy the CN balance condition (Equation 1) for all nodes of Srelated.

An 1pSolveAPI R package was used to solve the integer programming problem.

[Equation 2]
$Minimise {\sum }_{s ∈ {S}_{related}} ( μ ( {e}_{s} ( s ) ) - μ ( {e}_{r} ( s ) ) - {\sum }_{v ∈ {E}_{v} ( s )} μ ( v ) )$
subject to
$μ ( {e}_{s} ( s ) ) \geq μ ( {e}_{r} ( s ) ) + {\sum }_{v ∈ {E}_{v} ( s )} μ ( v )$
${\sum }_{v ∈ {E}_{v} ( s )} μ ( v ) \leq \vert μ ( {e}_{s} ( s ) ) - μ ( {e}_{s} ( {Adj}_{r} ( s ) ) ) \vert$
If siz (s) < 50 bp, μ(eₛ(s)) ∈ [0, max CN],
else if s is low-confident, μ(eₛ(s)) ∈ {CN(s), alternative CN(s)},
else, μ(eₛ(s)) = CN(s).

A first constraint prevented a nonsense solution whenever the adjacency exceeded the CN of the segment. A second constraint was related to an upper limit of the multiplicity of the SV edge, which did not exceed a difference between the multiplicities of adjacent segment edges at the SV breakpoint.

This preserved existing reference edges between adjacent segment edges as much as possible. Rarely, if the SV breakpoints were exactly reciprocal, the SVs may be filtered by the second constraint condition, and in order to restore this, a virtual segment (with a length of 0) between the reciprocal breakpoints remained.

Third to fifth constraints were related to the integer CN of the segment. When the CN can be measured because the segment size is too small or mis-segmentation due to SV breakpoint error occurs, the CN is replaced between 0 and maximum CN. Here, a minimum size threshold was set to 50 bp. In the case of a segment >50 bp, the multiplicity was fixed by an original estimate CN(s) if s was certain; otherwise, the multiplicity was changed within an alternative CN(s) range, which was an alternative CN range set to the next best integer state in ABSOLUTE in the current analysis. If there were multiple solutions, (1) the maximum multiplicity of the SV edge and (2) the multiplicity of the segment edge closest to the initial CN are selected. When the SV edge is maximized, actual SVs are recalled, but false SVs are still excluded (false SVs rarely meet the CN balance condition), and in the case of simple inversion and balanced translocation, a null solution in which the SV multiplicity is 0 is prevented. The solution may be found by gradually changing a boundary constraint condition for the SV and segment edges.

$${\sum }_{s ∈ {S}_{\text{?}}} μ ( {e}_{\text{?}} ( s ) ) > the mininum bound for SV edges$$

$${\sum }_{s ∈ {S}_{\text{?}}} \vert μ ( {e}_{\text{?}} ( s ) ) - CN ( s ) \vert < the maximum bound for segment edges$$

$$\text{?} \text{indicates text missing or illegible when filed}$$

In particular, SVs with the multiplicity of 0 are false-positives and are removed before the next iteration. The iteration step restarts with an SV set obtained after filtering the SVs with the multiplicity of 0. The value of the λ parameter of BIC-seq2 is used differently for the first and second iterations. Before setting the second iteration and the breakpoint graph, SV edges are added by remapping discordant and unmapped reads to de novo references of candidate neighbor items. In a somatic mode (with a control), germline variations is excluded and an additional process is performed to reconstruct a cancer genome graph into somatic SVs. In addition, SVs after constructing the breakpoints are classified into simple or complex SVs according to each breakpoint graph. Germline variants and short simple SVs (<100 kb) are bottlenecks in karyotype reconstruction because allele information for an allele-specific graph is not sufficient and over-segmentation of the genome may be caused.

Assuming negligible in a karyotype view, the breakpoint graph is simplified by removing the SV edges and CN bins for germline variants.

3. Measurement of Allele-Specific CN

In addition to integer CNs based on a total read depth, the read depth of heterozygous SNPs provides information on allele-specific CNs. In the breakpoint graph, the integer CN, μ(eₛ(s)) of each segment is divided into an allele-specific CN and ASCN(s) using a heterozygous SNP (if a control is present, all heterozygous SNPs from the control are used).

Let A={A1,A2, . . . , A_{[(μ(es(s))+1)/2]}} represents all possible states of allele-specific CNs of the genomic segment. Here, the integer CN is, if possible, divisible by a set of [(μ(eₛ(s))+1)/2], each Aᵢ={_{i,1,} A_{i,2}}, A_{i,1}+A_{i,2}=μ(eₛ(s)). For example, if the multiplicity μ(eₛ(s)) of the segment edge=3, there are two cases: A₁={0, 3} and A₂={1, 2}. If Aᵢ of each segment is given, a read depth oⱼ=(o_{j,1}, o_{j,2}) of the heterozygous SNP may be modeled using a negative binomial (NB) distribution when an allele-specific copy number for each SNP expressed as aⱼ=(a_{j,1}, a_{j,2}) is given. An SNP depth pair o_{j,1} and o_{j,2} is observed in a_{j,1} and a_{j,2}, respectively.

Here, the allele-specific copy number of the heterozygous SNP is a potential variable.

$$p ( O \vert Θ ) = {\prod }_{j = 1}^{N} {\sum }_{{a}_{j}} p ( {o}_{j} , {a}_{j} \vert b , p , {ϕ}_{1} , {ϕ}_{2} ) \qquad [ Equation 3 ]$$

$$p ( {o}_{j} \vert {a}_{j} , b , p , {ϕ}_{1} , {ϕ}_{2} ) = NB ( {o}_{j , 1} \vert b ( {pa}_{j , 1} + ( 1 - p ) ) , {ϕ}_{j , 1} ) NB ( {o}_{j , 2} \vert b ( {pa}_{j , 2} + ( 1 - p ) ) , {ϕ}_{j , 2} ) \qquad [ Equation 4 ]$$

When the purity p measured in the previous breakpoint graph construction is given, p(O|θ) per segment is maximized by estimating a haplotype base coverage b of a negative binomial distribution for A_{i,1} and A_{i,2}, respectively, and dispersion parameters ϕ₁ and ϕ₂. An EM algorithm is used to estimate a maximum likelihood parameter for the given Aᵢ.

A maximum likelihood \hat{L}(Aᵢ) and a likelihood score Score_{L}(Aᵢ) for each Aᵢ are obtained through iterative segmentation of μ(eₛ(s)), and an allele-specific copy number ASCN(s)=Â is obtained in the maximum likelihood situation.

$$\hat{L} ( {A}_{i} ) = P ( O \vert {\hat{Θ}}_{i} ) \qquad [ Equation 5 ]$$

$${Score}_{L} ( {A}_{i} ) = \frac{\hat{L} ( {A}_{i} )}{{\sum }_{j} \hat{L} ( {A}_{j} )} \qquad [ Equation 6 ]$$

$$\hat{A} = arg {max}_{{A}_{i}} \hat{L} ( {A}_{i} ) \qquad [ Equation 7 ]$$

Nevertheless, the initial estimate of ASCN(s) has uncertainty, and low confident ASCN(s) was defined as a case where (1) Score_{L}(Â)<0.8, or (2) the number of heterozygous SNPs <5. In the case of the low-confident segment, the present inventors searched for the best ASCN that minimized an objective function in the construction of an allele-specific breakpoint graph of the next round.

4. Construction of Allele-Specific Breakpoint Graph

Based on ASCN, an allele-specific breakpoint graph AG(S, E) was constructed, here, a node set S=S̅ ∪ S₁ ∪ S₂ consists of balanced S̅ and imbalanced nodes (S1 and S2 for temporal two haplotypes), which represent a head and a tail of a genomic segment with balanced and imbalanced ASCNs, respectively. In the allele-specific breakpoint graph, the imbalanced node is temporarily allocated to haplotype 1 or haplotype 2, whereas the balanced node is not allocated. The phased states (haplotype 1 and haplotype 2) of the imbalanced node are preserved within the imbalanced ASCN and may be converted in a genomic segment with the balanced ASCN.

Specifically, a genomic segment with an imbalanced ASCN, called an imbalanced AS segment, was represented by two head nodes S_{1,h} and S_{2,h} and tail nodes S_{1,t} and S_{2,t}. A genomic segment with balanced ASCN, called a balanced AS segment, was represented in the same manner as the breakpoint graph. Accordingly, the multiplicities of the segment edges for the imbalanced segment and the balanced segment were the ASCN and the total copy number, respectively. The allele-specific graph means that an SV edge may be allocated to one of the alleles if the multiplicity of the segment edges is imbalanced. In the case of the imbalanced AS segment, since a difference between adjacent segments varies depending on a temporal phased state of the AS segment, SV edges that uniquely align the imbalanced AS segment may be allocated to satisfy the copy balance condition. However, in the case of the balanced AS segment, the phased states of the AS segment and the SV edge were not uniquely determined because the value of an objective function does not depend on the phased state. The allele-specific breakpoint graph was constructed by the following integer programming problem. To adjust the multiplicity of the low-confident segment, a penalty function ε is added to the objective function to search all candidate integer divisions. This was proportional to the probability score rank of the integer division for the low-confident segment and was 0 for a confident segment. Since the penalty for the low-confident segment was added twice to the head node and the tail node for the segment, the penalty was divided by 2 while added to the objective function.

$$Minimise {\sum }_{s ∈ S} ( μ ( {e}_{s} ( s ) ) - μ ( {e}_{r} ( s ) ) - {\sum }_{υ ∈ {E}_{\text{?}} ( s )} μ ( υ ) + ε ( s ) / 2 ) \qquad [ Equation 8 ]$$

$$subject to$$

$$μ ( {e}_{s} ( s ) ) \geq μ ( {e}_{r} ( s ) ) + {\sum }_{υ ∈ {E}_{\text{?}} ( s )} μ ( υ )$$

$${\sum }_{υ ∈ {E}_{\text{?}} ( s )} μ ( υ ) \leq \vert μ ( {e}_{\text{?}} ( s ) ) - μ ( {e}_{s} ( {Adj}_{r} ( s ) ) ) \vert$$

$$If s is low - confident ,$$

$$\{ μ ( {e}_{s} ( {s}_{1} ) ) , μ ( {e}_{s} ( {s}_{2} ) ) \} ∈ \{ ASCN ( \hat{s} ) , alternative ASCN ( \hat{s} ) \} for s$$

$$∉ \overline{S} ,$$

$$μ ( {e}_{\text{?}} ( s ) ) = μ ( {e}_{\text{?}} ( \hat{s} ) ) for s ∈ \overline{S} ,$$

$$else ,$$

$$\{ μ ( {e}_{\text{?}} ( {s}_{1} ) ) , μ ( {e}_{\text{?}} ( {s}_{2} ) ) \} = ASCN ( \hat{s} ) for s ∉ \overline{S} ,$$

$$μ ( {e}_{\text{?}} ( s ) ) = μ ( {e}_{\text{?}} ( \hat{s} ) ) for s ∈ \overline{S} .$$

$$\text{?} \text{indicates text missing or illegible when filed}$$

When the ASCN measurement of the node Ŝ is represented by ASCN(ŝ)={ASCN₁(ŝ), ASCN₂(ŝ)}, an integer programming problem (wherein s of the objective function may be s1 and s2 for an imbalanced AS segment) requires exponential time for μ(eₛ(s₁)) and μ(eₛ(s₂)) to replace ASCN₁(ŝ) and ASCN₂(ŝ), or ASCN₂(ŝ) and ASCN₁(ŝ). Here, ŝ represents a node of the previous breakpoint graph, which may be extended to s1 and s2 or remain as is when ASCN(ŝ) is balanced.

To solve the integer programming problem in a series of imbalanced AS segments, the multiplicity of the segment edges were determined using a heuristic.

5. Haplotype Segments

Haplotype segments H={H₁, H₂, . . . , Hₙ} are defined from the allele-specific graph AG(S, E), and each element Hi={H_{i,1}, H_{i,2}} is an imbalanced AS segment set for haplotype 1 and haplotype 2, wherein heterozygous SNPs are phased by (1) allelic imbalances and (2) local (<1 Mb) nonhomologous SVs. First, consecutive imbalanced AS segment sets was collected between balanced AS segments in an allele-specific graph, wherein the phases of the imbalanced AS segments of each set were determined by integer programming of the previous allele-specific breakpoint graph construction section. Then, the SVs between the segments of the imbalanced AS segments of two sets were classified into homologous (>100 bp homology) and nonhomologous SVs (≤100 bp homology), and then the presence of nonhomologous SVs between the imbalanced AS segments was confirmed. Except for a rare possibility that homologous chromosomes are exchanged by a homologous mechanism at the same focal breakpoint, focal nonhomologous SV was assumed to occur in a single allele. The assumption simplified the haplotype phase problem by preventing false allelic transitions (haplotype transition errors), and sequences of the imbalanced AS segments were merged into haplotype segments.

When two sequences of the imbalanced segment, i.e., k, k+1, . . . , k+k′ segments and 1, 1+1, . . . , 1+1′ segments, exist and there are nonhomologous SVs between the segments, haplotype segments were defined as follows.

H_{i,1}={(h_{1,h}^{k}, h_{1,t}^{k}), . . . , (h_{1,h}^{k+k′}, h_{1,t}^{k+k′}), (h_{1,h}^{l}, h_{1,t}^{l}), . . . , (h_{1,h}^{l+l′}, h_{1,t}^{l+l′})} [Equation 9]

H_{i,2}={(h_{2,h}^{k}, h_{2,t}^{k}), . . . , (h_{2,h}^{k+k′}, h_{2,t}^{k+k′}), (h_{2,h}^{l}, h_{2,t}^{l}), . . . , (h_{2,h}^{l+l′}, h_{2,t}^{l+l′})} [Equation 10]

The head and tail node pair (h_{1,h}^{k}, h_{1,t}^{k}) represents a k-th segment of haplotype 1, which was allocated based on the node of the allele-specific graph (s_{1,h}^{k}, s_{1,t}^{k}) or (s_{2,h}^{k}, s_{2,t}^{k}). The allocation is determined by the integer programming for the copy number balance condition of Hi together with the constraint condition for nonhomogeneous SVs. The SNP of the haplotype segment is phased by maximizing the possibility of Equation 4 when considering the aligned state of the imbalanced AS segments in the haplotype segment. When oᵢ,1 is observed in snp_{j,1} of the k-th segment of Hᵢ, the heterozygous SNPs, snp_{j,1} and snp_{j,2} correspond to H_{i,1} and H_{i,2}, respectively
p((o_{j,1}, o_{j,2})|(μ(eₛ(h_{1,h}^{k})), μ(eₛ(h_{2,h}^{k}))), Θₖ)>p((o_{j,2}, o_{j,1})|(μ(eₛ(h_{1,h}^{k})), μ(eₛ(h_{2,h}^{k}))), Θₖ)).

6. Construction of Haplotype Breakpoint Graph

Previously, in the allele-specific breakpoint graph, sequences of imbalanced AS segments and nonhomogeneous SVs defined a haplotype segments H and the phase of the heterozygous SNP was adjusted in the imbalanced ASCN of the haplotype segment. The haplotype breakpoint graph was constructed by phasing SNPs in balanced AS segments using population information and determining the end-to-end order of haplotype segments. Haplotypes were obtained using a restricted version of a Viterbi algorithm for a hidden Markov model (HMM) of BEAGLE, wherein the transition and emission probabilities were defined in a localised haplotype-cluster graph. Since imbalanced heterozygous SNPs were already staged in haplotype segments, the Viterbi path was run to follow the phased order of SNPs. The Viterbi path phased the heterozygous SNPs in balanced ASCNs while determining the order of the haplotype segments. Finally, the haplotype graph HG(S, E) was constructed. The present inventors expressed haplotype-specific copy numbers for the node Ŝ as HSCN₁(ŝ) and HSCN₂(ŝ) from the allele-specific graph, which were obtained through haplotype phasing. In the case of an imbalanced node, depending on the phased state of the heterozygous SNP, HSCN₁(ŝ) and HSCN₂(ŝ) are μ(eₛ(ŝ₁)) and μ(eₛ(ŝ₂)) or μ(eₛ(ŝ₂)) and μ(eₛ(ŝ₁)). In the case of the balanced node, HSCN₁(ŝ₁)=HSCN₂(ŝ)=μ(e(ŝ))/2. After obtaining the haplotype specific copy number, a haplotype graph was constructed by the following integer programming. That is, in the imbalanced node, the multiplicity of segment edges was aligned with the extension of the balanced node in the allele-specific graph, and the multiplicities of the reference and variant edges were allocated to minimize the objective function.

$$Minimise {\sum }_{s ∈ S} ( μ ( {e}_{s} ( s ) ) - μ ( {e}_{r} ( s ) ) - {\sum }_{υ ∈ {E}_{υ} ( s )} μ ( υ ) ) \qquad [ Equation 11 ]$$

$$subject to$$

$$μ ( {e}_{s} ( s ) ) \geq μ ( {e}_{r} ( s ) ) + {\sum }_{υ ∈ {E}_{\text{?}} ( s )} μ ( υ )$$

$${\sum }_{υ ∈ {E}_{\text{?}} ( s )} μ ( υ ) \leq \vert μ ( {e}_{s} ( s ) ) - μ ( {e}_{s} ( {Adj}_{r} ( s ) ) ) \vert$$

$$μ ( {e}_{s} ( {s}_{1} ) ) = {HSCN}_{1} ( \hat{s} )$$

$$μ ( {e}_{s} ( {s}_{2} ) ) = {HSCN}_{2} ( \hat{s} )$$

$$\text{?} \text{indicates text missing or illegible when filed}$$

7. Enumeration of Eulerian Paths

To identify candidate genomes, Eulerian paths were enumerated by alternating segment edges and SV/reference edges in the haplotype graph constructed in the previous step.

Head and tail nodes that did not meet a copy number balance condition (including original telomere ends) were considered as the ends of the reconstructed chromosome P, which may also be ends or breakages due to missed SVs or miscalculated CNs. A circular chromosome C included in the DM cluster was observed in a circular path. An Eulerian decomposition problem (EDP) was defined to find linear and circular chromosomes in the breakpoint graph. The min-EDP, |P|+|C|, which minimizes the number of paths and cycles, has previously been proposed to describe the most probable karyotype, but the min-EDP is not always biologically relevant (i.e., max-EDP may be applicable). In this study, the enumeration of minimum entropy Eulerian paths that prioritized the decomposition of P and C with minimum entropy was formulated. To enumerate candidate Eulerian paths, each tree node used a multiway tree structure representing a pairing state of a breakpoint graph edge. The multiway tree was extended in a root-to-leave model by sequentially increasing the level and processing of each node in the breakpoint graph. A leaf node represents an available edge pairing state that describes the Eulerian path to reach all genomic segments.

The enumeration of all the Eulerian paths is an NP-hard problem, and an Eulerian path with minimum entropy is prioritized as the biologically relevant case. First, linked chromosomes were separated and the enumeration was performed inside the linked chromosomes. In the case of highly segmented genomes, the haplotype breakpoint graph may be simplified by further excluding simple SVs (tandem duplications, deletions, and block-exchange insertions) that do not significantly affect the candidate karyotypes. Thereafter, the minimum entropy search was applied to designate the priority of the solution using the minimum entropy. For example, a node in the multiway tree at a level 1 represents an edge pairing state of a total of 1 nodes in the breakpoint graph, and there are n unique paths. The total number of paths is w=w₁+w₂+ . . . +wₙ, wherein wᵢ represents the multiplicity of an i-th path. The entropy at levels 1 and e₁ is derived from the following Equation:

$${e}_{l} = - {\sum }_{i}^{n} ( {w}_{i} / w ) log ( {w}_{i} / w ) \qquad [ Equation 12 ]$$

The low entropy indicates that the SV sets were duplicated through additional amplification processes such as arm-level duplication, WGD and other duplication processes in HSR and DM. This required a shorter distance than individual occurrence of SVs. When the solution space grows too fast, branches are cut from the multiway tree and a candidate genome was obtained from the leaf nodes of the remaining branches.

8. Construction of Breakpoint Graphs for Multi-Sample Data

For multiple samples, a SV set was integrated from the initial SV of each sample. A breakpoint graph of each sample was constructed using the integrated SV set. Then, SVs were classified into private and shared SVs based on the presence of raw SV evidence (discordant or split reads). If the shared SVs are not called in primary tumors, the private SVs were observed in metastatic tumors and vice versa. This approach of using the integrated SV set has an advantage of adding SV edges that are not called at each sample to the graph when the supported copy number depth and adjacent SV information are present in the corresponding sample. When there was difficulty in distinguishing the private SV and the shared SV, iteration optimization was performed once again.

9. Simulated Data Set

First, 12 simulated normal tumor pairs were generated from subjects NA12878, HG00732, NA19238 and HG00513 in phase 3 of the 1000 Genomes Project. The present inventors simulated approximately 3000 germate and 200 somatic SVs per cancer genome, where the proportion and size of each SV type was derived from previous studies. The diploid to tetraploid cancer genomes were generated by the WGD operation and each cancer genome was mixed with normal genomes matched with different purities (60%, 75% and 90%). Generate Illumina HiSeq 2×100 reads were generated from heterogeneous genomes using 3×, 5×, 10×, 15×, and 20× haplotype fold ranges using the ART (version 2.5.8) and mapped to a GRCh37 reference genome using Burrows-Wheeler Aligner-Maximal Exact Matches (BWA-MEM). To compare the performance according to a human reference genome version, the same simulation scheme was used to generate diploid to tetraploid cancer genomes in NA12878 based on the GRCh38 reference and the read values were mapped to the GRCh38 reference genome.

10. Data Collection and Preprocessing

For data collection, WGS and RNA-seq data of a HeLa cell line and WGS data of a lung cancer cell line were downloaded using the SRA Toolkit (version 2.8.2). A GDC client (version 1.2.0) was used to download the WGS data of a TCGA sample. An EGA client (version 2.2.2) was used to download WGS data of metastatic breast cancer. In paired-end and mate-pair libraries of the HeLa genome, the reads were mapped to the human reference genome GRCh37 using BWA-MEM with basic parameters (version 0.7.15). DELLY2, Manta and NovoBreak were used for SV calls in paired-end data, and DELLY2 was used for mate-pair data. Initial SV calls from two libraries were merged into the input of InfoGenomeR and paired-end data were used for CN calls and allele-specific and haplotypic estimation. SNPs were detected using BCFtools (version 1.3). Values read from three libraries of HeLa transcripts were mapped and quantified using HISAT2 and CuffLinks, and mean counts were collected in duplicates to measure expression values. The WGS data of the lung cancer cell line, the TCGA sample and the metastatic breast cancer included preprocessed data (BAM) mapped to GRCh37 and variants were called in the same manner as paired-end HeLa and simulated data sets.

<Discussion>

Our graph-based framework, InfoGenomeR, integrates individual variant calling for SV and CNA, purity and ploidy measurement, and haplotype estimation. Based on a breakpoint graph, InfoGenomeR generates a haplotype graph to narrow a target genome according to allele and haplotype specific information. Consequently, the karyotype of the target genome is specialized by increasing the range of individual variant calling and facilitating the identification of genome-whole SVs.

InfoGenomeR is used to identify complex rearrangement topologies HSR, DM, HSR/DM and CT in reconstructed cancer genome karyotypes. In previous studies, the identification of DM was performed using integrated SVs and CNAs, but the analysis was limited to locally amplified regions without recovering the haplotypic karyotypes. ShatterSeek identified CT using an integrated approach of SV and CNA. However, a karyotype structure such as a derivative chromosome and DM due to CT was not provided. Recently, a decomposition method of DM and/or linear chromosomes based on a haplotype graph has been introduced. Nevertheless, this method lacks interpretation for other topologies such as HSR, HSR/DM or CT. JaBbA introduced a complex topology with DM, but excluded other karyotypes that may be derived from the reconstructed haplotype. When InfoGenomeR is used, complex topologies may be simultaneously understood through karyotype reconstruction at the whole genome level, as shown in the analysis of TCGA (FIG. 4) and EGA data (FIG. 5). InfoGenomeR may help to identify recurrent derivative chromosomes generated on chromosomes 11 and 17 together with the HSR of BRCA. The analysis for SV clusters of the present inventors showed that CCND1 and ERBB2 were often clustered closely to these derivative chromosomes. Furthermore, the present inventors found that GBM and OV were mainly characterized by HSR/DM or DM and HSR and by fold-back inversions for other chromosomes.

CT was recently reported by ShatterSeek to be found in more than half of cancers, and CT with other complex events is more prevalent than standard CT showing oscillation patterns between two CN states. However, a target of ShatterSeek was limited to identifying SV clusters in CT, and there is no reconstitution strategy, so that the structure of the derivative chromosome is not comprehensively examined. The result of the present inventors showed CT-related HSR, HSR/DM or DM topologies in the chromosomal structure by reconstructing the cancer genome karyotype. The present inventors showed that chromosome 17 was a template chromosome, and repeatedly rearranged with other chromosomes with CT in BRCA, so that a complex event with CT on many chromosomes produced a derivative chromosome. It has been proposed that a CT-related complex event in the formation of the derivative chromosome contributed to the amplification of cancer-related genes such as CCND1, CDK4, MDM2 and ERBB2. The result of the present invention showed that cancer-related genes were amplified in the formation of the derivative chromosome. Overall, the present inventors provided perception of the karyotype aspect of complex rearrangements associated with CT.

Through multi-sample analysis, the present inventors were able to identify the evolution process of HSR and DM generation with CT during metastatic tumor evolution. Previously, SVs were examined as appearing in metastasis. However, the distinction between private SVs and shared SVs was not clear and karyotypic characterization was not performed. The present inventors observed that although SVs may be identified as shared SVs based on CNA evidence present in primary and metastatic tumors, the SVs may be misidentified as private SVs with conventional SV calling approaches. While constructing the breakpoint graph, the present inventors performed imputation on candidate shared SVs that may be present in both primary and metastatic tumors to clearly distinguish true private SVs. These private SVs were displayed together in HSR and DM topologies with CT (FIGS. 5D and 5E). The present inventors characterized the karyotype by constructing the derivative chromosome to provide a basis for a structure-based analysis of tumor evolution. Nevertheless, there were limitations in the current analysis. First, the breakpoint graph was adjusted in an intermediate step during iteration optimization, but our applications for primary and metastatic tumors were independent of each other. In addition, although subclonal SV or CNA could elucidate the tumor evolution process, a clone-specific interpretation was not performed. A common approach to subclones through multiple samples is required for future analysis.

Despite the successful application of InfoGenomeR for the construction of the whole genomic karyotypes, there are clear opportunities to improve and expand this framework in the future. For example, in each iteration of the optimization procedure, potential false SVs were filtered and not added to the graph except for an intermediate SV addition step (b of FIG. 1). However, this may prevent the recall of the actual SVs. In addition, some translocations were not called in the initial SV calls of the HeLa cell line, which may occur in centrosomes or unmappable iteration regions. Long read sequencing is required to find SVs that cannot be identified by short read sequencing. Our framework may obtain an advantage of long read sequencing technique by integrating long read SV calls into SV calls of short read sets. In addition, the current framework constructed a representative graph for dominant tumor cells from large WGS data to remove minor changes in CN during the optimization procedure. However, a small number of subclonal populations may have been removed during this process. Moreover, this removal may be problematic for samples where the subclonal population forms a significant population (wherein, the present inventors focused on non-clonal samples with cancer purity of 70% or greater). When deconvolution methods for CNA and SV are integrated into the framework, the examination of the subclonal structure is additionally permitted and multiple breakpoint graphs thereof are generated. Finally, graph-based reconstruction of multi-sample genomes started to provide the basis for structure-based analysis. The present inventors propose that a phylogenetic method for examining karyotype evolution (i.e., measuring edit distances in the breakpoint graphs) is required. InfoGenomeR is not limited to cancer, but may also be used for other genetic diseases. A potential application is a somatic mutation assay in neurological diseases that contribute to the genetic diversity of human neurons, such as somatic SNV. Somatic SV has not been comprehensively examined in neurological diseases, but fine abnormalities have emerged. The application of InfoGenomeR may detect genetic variations which are not found in SNV.

In summary, the present inventors developed a method for reconstructing cancer genome karyotypes and searched multi-sample data using karyotypes of complex SVs and primary and metastatic cancer cells from three cancer types BRCA, GBM and OV. More cancer types should be searched to determine wider karyotype changes occurring during cancer development and evolution. The present inventors expect that oncogenic genes of these complex SVs may be used to identify clinical therapeutic candidates.

In this specification, the present disclosure has been mainly described with reference to limited embodiments, but various embodiments are possible within the spirit scope of the present disclosure. In addition, although not described, it will be understood that equivalent means are also combined as they are in the present disclosure. Therefore, the scope of the present disclosure should be determined by the appended claims.

What is claimed is:

1. A genome reconstruction method using whole genome data comprising steps of:
1) detecting an initial structural variation of a genomic segment of a whole genome sequence;
2) constructing a breakpoint graph from the genomic segment and the structural variation;
3) constructing an allele-specific breakpoint graph;
4) constructing a haplotype breakpoint graph; and
5) enumerating Eulerian paths by pairing edges of the breakpoint graph.

2. The genome reconstruction method of claim 1, wherein in step 1), the structural variation is indicated as head-to-head (HH), head-to-tail (HT), tail-to-head (TH) or tail-to-tail (TT) according to a direction of breakpoint adjacencies.

3. The genome reconstruction method of claim 1, wherein in step 2), graph nodes include a head node (Sₕ) and a tail node (Sₜ), and graph edges include a segment edge (Eₛ), a reference edge (Eᵣ), and an SV edge (Eᵥ).

4. The genome reconstruction method of claim 2, wherein the segment edge links a head node and a tail node of an nth genomic segment, and the multiplicity of the segment edge indicates the copy number (CN) of the genomic segment.

5. The genome reconstruction method of claim 2, wherein the reference edge links an nth tail node and a n+1th head node between the nth and n+1th genomic segments, and represents adjacency between adjacent genomic segments present in the reference genome.

6. The genome reconstruction method of claim 2, wherein the SV edge represents adjacency between genomic segments which are not present in the reference genome.

7. The genome reconstruction method of claim 1, wherein step 2) is performed by the following iterative steps:
a) performing local copy number segmentation;
b) predicting an integer copy number (CN) by integer programming; and
c) determining edge multiplicity by the integer programming.

8. The genome reconstruction method of claim 7, wherein the a) performing of the local copy number segmentation includes determining a breakpoint consisting of the following two terms:
a likelihood term describing how well a model with breakpoints fits read-depth data; and
a parameter or penalty term of controlling the number of breakpoints and preventing over-segmentation.

9. The genome reconstruction method of claim 7, wherein the b) predicting of the integer copy number includes sequentially substituting the integer copy number according to a high probability in an integer measurement model from the read-depth of the genomic segment.

10. The genome reconstruction method of claim 7, wherein the edge multiplicity is indicated by the multiplicities of a segment edge, a structural variation edge, and a reference edge.

11. The genome reconstruction method of claim 7, further comprising:
d) removing a structural variation with edge multiplicity of 0.

12. The genome reconstruction method of claim 11, further comprising:
iteratively performing steps a) to d) until the structural variation with the edge multiplicity of 0 is not detected.

13. The genome reconstruction method of claim 1, wherein step 3) further includes dividing an integer CN into an allele-specific copy number (ASCN) by integer programming.

14. The genome reconstruction method of claim 13, wherein the dividing of the integer CN into the allele-specific copy number (ASCN) by the integer programming is performed using a negative binomial model for different depths of the SNP.

15. The genome reconstruction method of claim 1, wherein the allele-specific breakpoint graph is constructed based on the allele-specific copy number (ASCN).

16. The genome reconstruction method of claim 1, wherein the allele-specific breakpoint graph consists of a balanced node and an imbalanced node.

17. The genome reconstruction method of claim 1, wherein the 4) constructing of the haplotype breakpoint graph includes
defining a haplotype segment from the allele-specific breakpoint graph of step 3);
phasing balanced heterozygous SNP and imbalanced heterozygous SNP; and
constructing a haplotype breakpoint graph by integer programming.

18. The genome reconstruction method of claim 1, wherein the enumerating of the Eulerian paths includes pairing breakpoint graph edges using a multiway tree structure.

19. The genome reconstruction method of claim 18, wherein the enumerating of the Eulerian paths includes prioritizing an Eulerian path with minimum entropy.
